(19) United States
(12) Patent Application Publication
(10) Pub. No.: US 2010/0248346 A1
(43) Pub. Date: Sep. 30, 2010

(54) ANALYZER

(75) Inventors: Shuhei KANEKO, Kobe-shi (JP); Kazuya Fukuda, Kobe-shi (JP); Hidenari Takaoka, Kobe-shi (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION, Kobe-shi (JP)

(21) Appl. No.: 12/721,983

(22) Filed: Mar. 11, 2010

(30) Foreign Application Priority Data
Mar. 31, 2009 (JP) 2009-085060

(51) Int. Cl. G01N 33/48

(52) U.S. Cl. 435/287.1; 422/68.1

(57) ABSTRACT

An analyzer comprising: a housing comprising a first space therein; a reagent accommodation section, disposed in the housing, comprising a second space therein for accommodating a reagent container containing a reagent, and an air induction port for inducting air from the first space to the second space; and a cooler for cooling the air which has been inducted from the first space through the air induction port into the second space, is disclosed.

### FIELD OF THE INVENTION

The present invention relates to an analyzer for analyzing a sample using a reagent held in a reagent container such as a blood coagulation analyzer, immunoanalyzer and the like.

### BACKGROUND

There are known conventional analyzers for analyzing the measurement results of a measurement sample prepared by mixing a specimen and a reagent. In such analyzers, a reagent container holding the regent is stored in a predetermined reagent reservoir, and the interior of the reagent reservoir is cooled to a predetermined temperature to prevent degrading of the reagent. For example, Japanese Laid-Open Patent Publication No. 2006-84366 discloses an automated analyzer provided with a reagent refrigeration section having a reagent accommodation section that accommodates a plurality of reagent containers, a cold air inductor provided adjacent to the regent accommodation section and for introducing the cold air from the cooler, a cold air circulation unit having a cold air discharger for returning the cold air to the cooler, a cold air inductor inlet for introducing the cold air from the cold air inductor to the reagent accommodation section, a cold air discharge outlet for expelling the cold air from the reagent accommodation section to the cold air discharger, and an outside-air inlet disposed near the cooler of the cold air discharger for introducing outside air into the cold air discharger.

This automated analyzer, however, is configured to take in outside air from the outside-air inlet into the reagent refrigeration section so as to eliminate the air pressure differential within the reagent accommodation section and suitably circulate the cold air. Therefore, there is concern that excessive outside air may flow from the outside air inlet when the laboratory in which this automated analyzer is installed has an unstable airflow due to, for example, the operation of air conditioners, fans and the like. Problems arise when warm outside air flows excessively from the outside-air inlet inasmuch as the water vapor contained in the outside air comes into contact with the reagent containers and the like and large amount of dew condensation occurs so as to have a high probability of adversely affecting the reagent.

### SUMMARY OF THE INVENTION

The scope of the present invention is defined solely by the appended claims, and is not affected to any degree by the statements within this summary.

A first aspect of the present invention is an analyzer comprising: a housing comprising a first space therein; a reagent accommodation section, disposed in the housing, comprising a second space therein for accommodating a reagent container containing a reagent, and an air induction port for inducting air from the first space to the second space; and a cooler for cooling the air which has been inducted from the first space through the air induction port into the second space.

### BRIEF DESCRIPTION OF THE DRAWINGS

FIG. 1 is a perspective view showing the general structure of a first embodiment of the sample analyzer of the present invention;

FIG. 2 is a top view of the sample analyzer shown in FIG. 1;

FIG. 3 is a top view of the measuring device of the sample analyzer shown in FIG. 1;

FIG. 4 is a perspective view of the inside of the measuring device and the reagent storage section;

FIG. 5 is a top view of the inside of the measuring device and the reagent storage section shown in FIG. 4;

FIG. 6 is a block diagram of the control device of the sample analyzer shown in FIG. 1;

FIG. 7 is a perspective view showing an example of a first reagent container rack;

FIG. 8 is a perspective view showing an example of a second reagent container rack;

FIG. 9 is a perspective view showing the reagent container held in the first reagent container rack shown in FIG. 7;

FIG. 10 is a perspective view showing the reagent container held in the second reagent container rack shown in FIG. 8;

FIG. 11 is a block diagram of the sample analyzer shown in FIG. 1;

FIG. 12 is a block diagram of the controller of the measuring device of the sample analyzer shown in FIG. 1;

FIG. 13 is a perspective view showing the air circulation unit of the reagent storage section shown in FIG. 4;

FIG. 14 is a cross sectional view schematically showing the reagent storage section shown in FIG. 4;

FIG. 15 is a perspective view of the sample analyzer of FIG. 1 viewed from the back side;

FIG. 16 is a bottom view of the sample analyzer shown in FIG. 1; and

FIG. 17 is a brief perspective view showing the housing of the sample analyzer shown in FIG. 1.

### DETAILED DESCRIPTION OF THE PREFERRED EMBODIMENTS

The preferred embodiments of the present invention are described hereinafter with reference to the drawings.

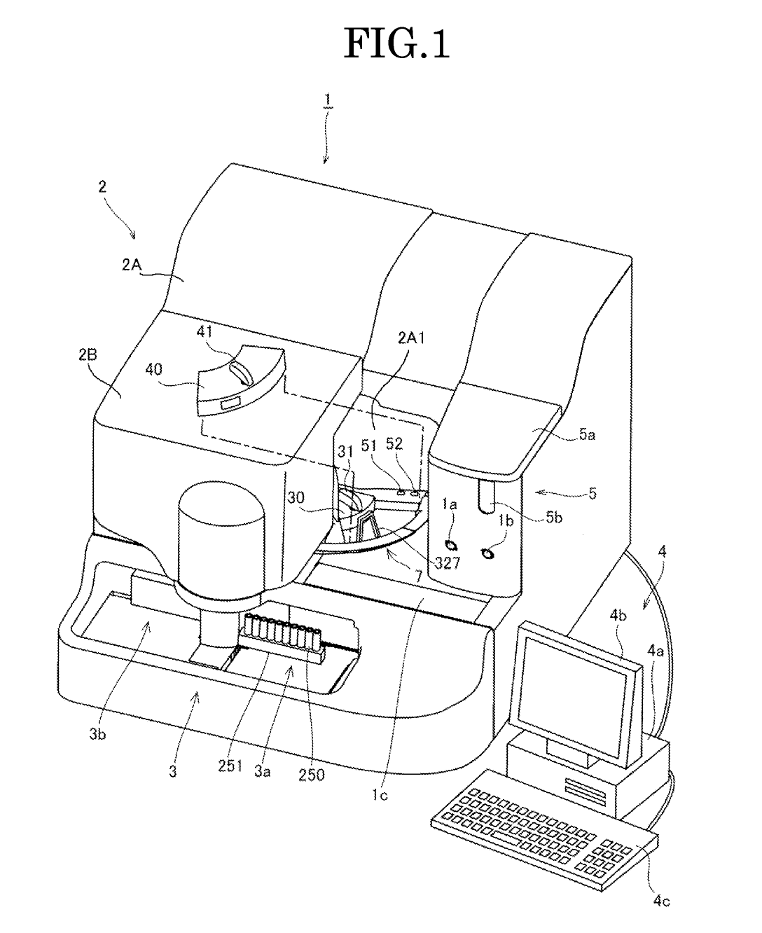

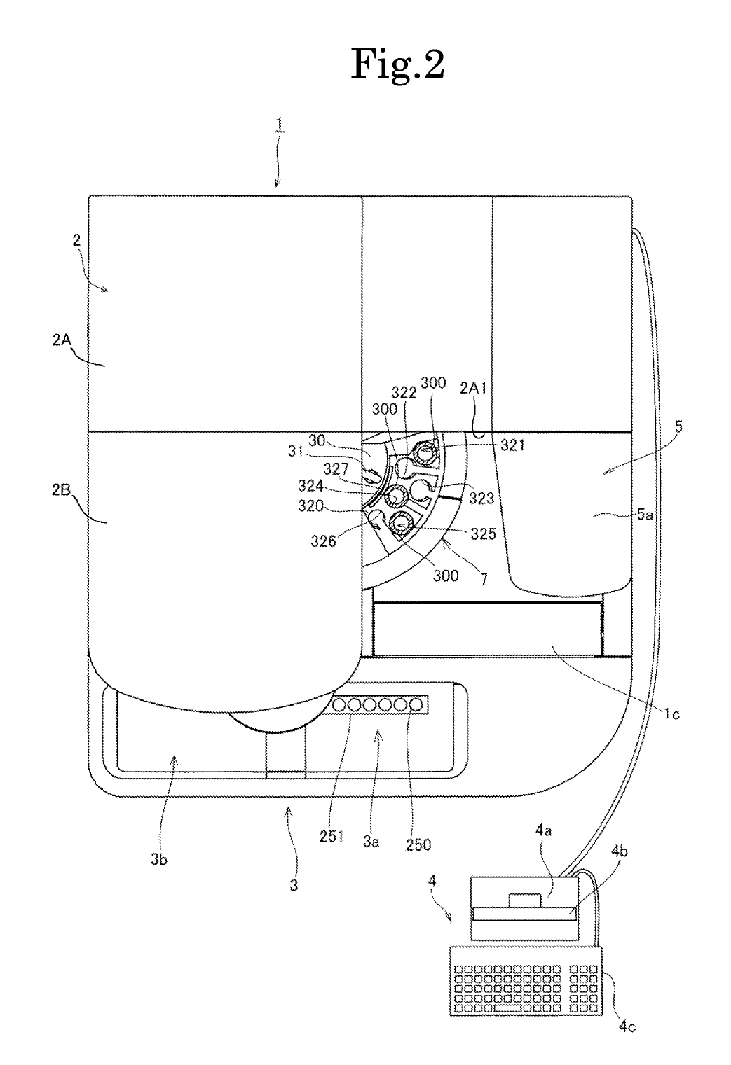

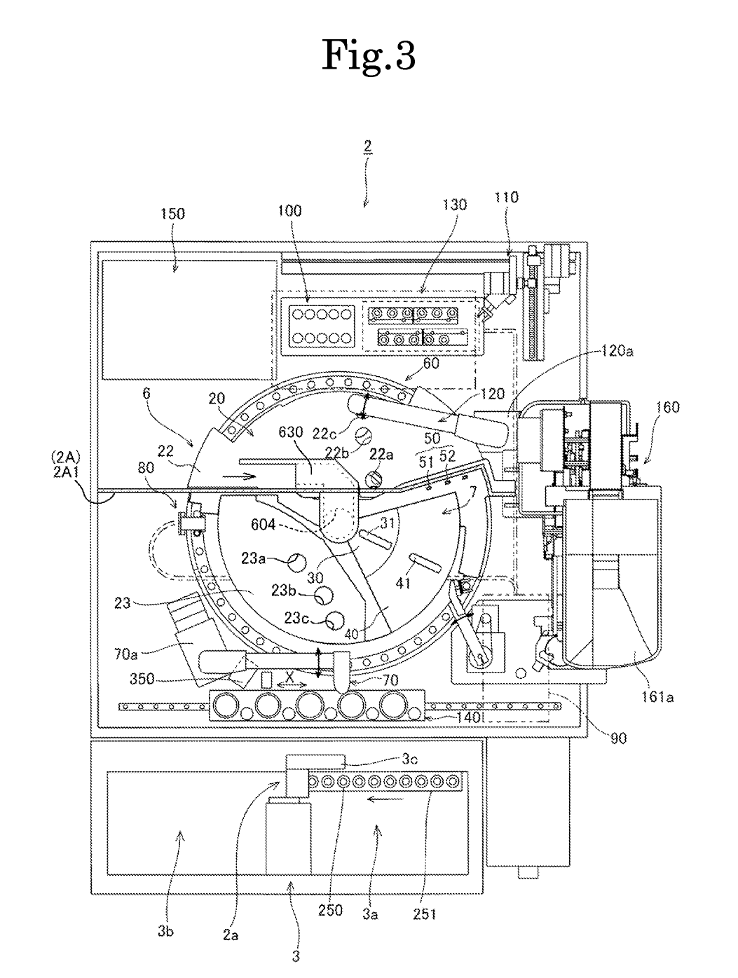

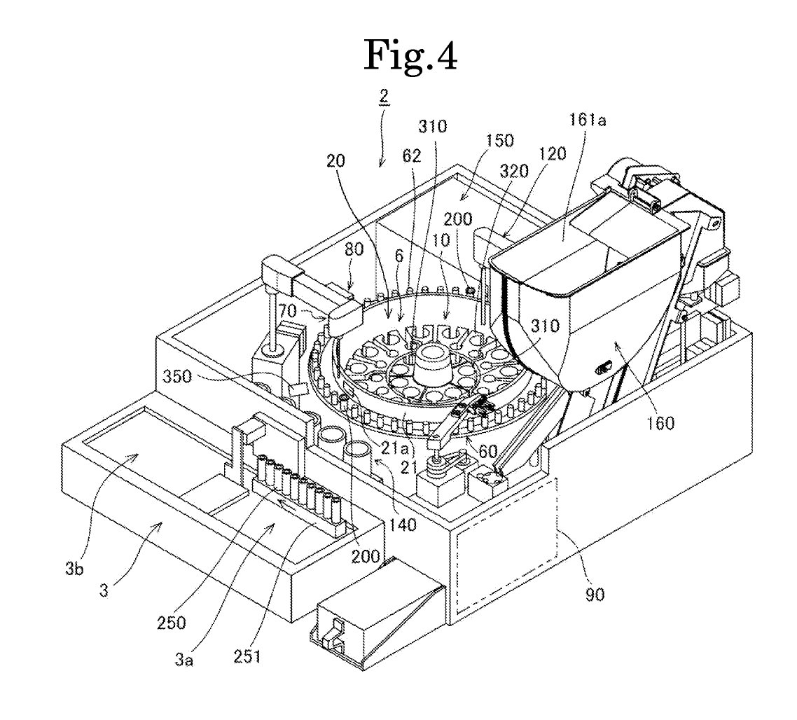

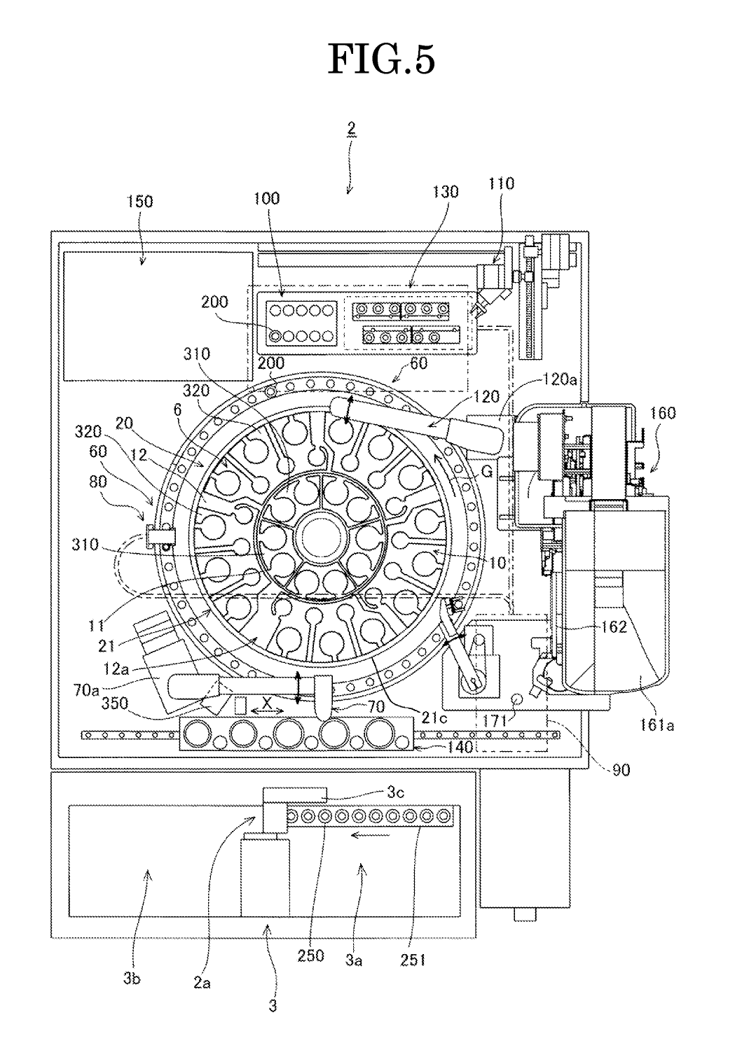

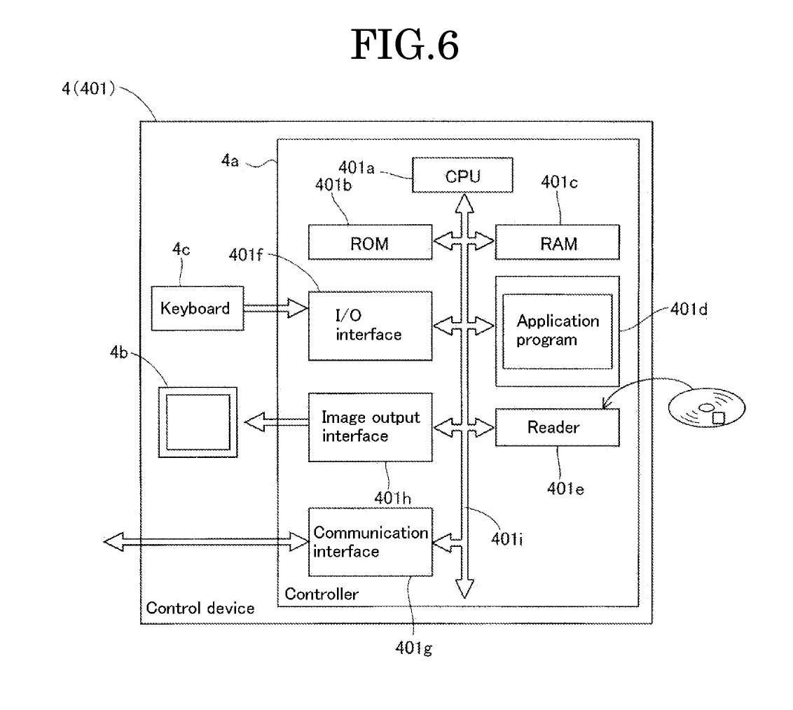

FIG. 1 is a perspective view showing the general structure of the sample analyzer 1 of the first embodiment of the present invention, and FIG. 2 is a top view showing the general structure of the sample analyzer 1. FIG. 3 is a top view of the measuring device of the sample analyzer shown in FIG. 1. FIG. 4 is a perspective view showing the inside of the measuring device and the reagent storage section, and FIG. 5 is a top view showing the inside of the measuring device and the reagent storage section shown in FIG. 4. FIG. 6 is a block diagram showing the control device of the sample analyzer 1.

[General Structure of Sample Analyzer 1]

The sample analyzer 1 is a device for optically measuring and analyzing the amount and degree of activity of specific substances related to blood coagulation and fibrinolytic functions, and uses blood plasma as the sample. The sample analyzer 1 of the present embodiment optically measures a sample using the blood coagulation time, synthetic substrate, and immunoturbidity methods. The blood coagulation time used in the present embodiment is a measurement method for detecting the process of sample coagulation as a change in light transmittance. Measurement criteria include, PT (prothrombin time), APTT (activated partial thromboplastin time), and Fbg (fibrinogen quantity) and the like. Measurement criteria of the synthetic substrate method include ATIII and the like, and those of the immunoturbidity method include D-dimer, FDP and the like.

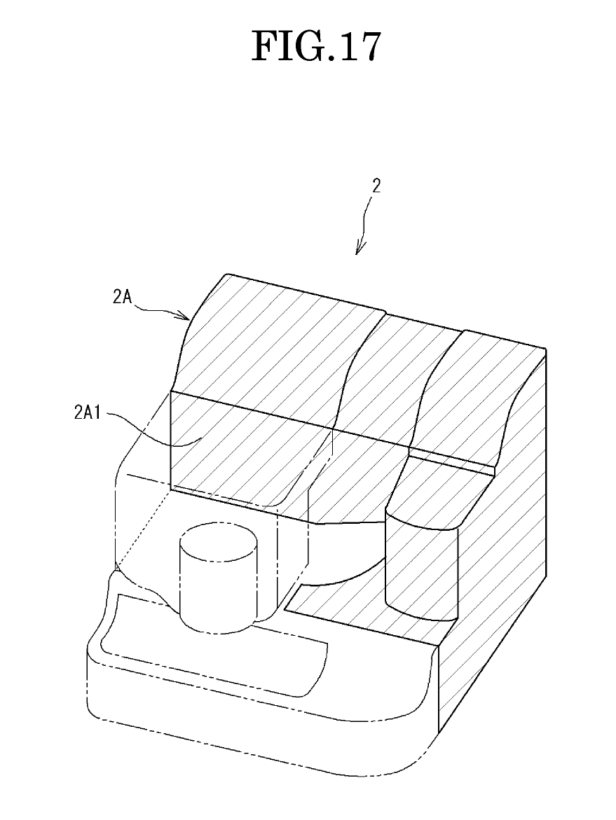

As shown in FIGS. 1 and 2, the sample analyzer 1 is configured by a measuring device 2, transporting device 3 disposed on the front side of the measuring device 2, and a control device 4 that is electrically connected to the measuring device 2. The measuring device 2 is also covered by a housing 2A and a cover body 2B. The housing 2A is indicated by the diagonal lines in FIG. 17; the housing covers the backside and bottom side of the measuring device 2 and provides interior space. The cover body 2B is mounted on the top front left side of the housing 2A so as to cover the front left side of the measuring device 2 in a manner as to be openable. The measuring device 2 is also provided with a cuvette acceptor 5 for receiving cuvettes 200 (refer to FIG. 4) that accommodate a sample to be subjected to measurements. The cuvette acceptor 5 is provided with a cover 5a that can be opened and closed, and a window 5b through which the interior of the cuvette acceptor 5 can be viewed. Furthermore, an urgent stop button 1a, and measurement start button 1b are provided on the front side of the cuvette acceptor 5. The cover 5a (refer to FIG. 1) is provided for accepting a cuvette 200 in a first hopper 161a (refer to FIG. 4) of a cuvette supplying device 160. A user can verify the remaining quantity of cuvettes 200 retained in the first hopper 161a (refer to FIG. 4) through the window 5b. The urgent stop button 1a (refer to FIG. 1) has the function of stopping a measurement under urgent circumstances. The measurement start button 1b (refer to FIG. 1) is configured to start a measurement when pressed. Thus, a user can immediately start a measurement after loading the cuvettes 200. Note that measurements may also be started and stopped by an operation of the control device 4.

[Control Device 4 Structure]

The control device 4 is configured by a personal computer 401 (PC), and includes a controller 4a, display 4b, and keyboard 4c, as shown in FIGS. 1 and 2. The controller 4a has the functions of sending the operation start signal of the measuring device 2 to the controller 501 of the measuring device 2, and analyzing the optical information of the sample obtained by the measuring device 2. The controller 4a is configured by a CPU, ROM, RAM and the like. Furthermore, the display 4b is provided to display information relating to interference substances (hemoglobin, bilirubin, chyle (fats)) present in a sample, and analysis results obtained by the controller 4a.

As shown in FIG. 6, the controller 4a is mainly configured by a CPU 401a, ROM 401b, RAM 401c, hard disk 401d, reading device 401e, I/O interface 401f, communication interface 401g, and image output interface 401h. The CPU 401a, ROM 401b, RAM 401c, hard disk 401d, reading device 401e, I/O interface 401f, communication interface 401g, and image output interface 401h are mutually connected via a bus 401i.

[Transporting Device 3 Structure]

As shown in FIGS. 1 through 3, the transporting device 3 has the function of transporting a rack 251 holding a plurality of test tubes 250 (in the present embodiment, 10 test tubes) containing samples to the aspirating position 2a (refer to FIG. 3) of the measuring device 2 so as to supply the samples to the measuring device 2. The transporting device 3 has a rack placement region 3a for placing the racks 251 holding the test tubes 250 containing unprocessed samples, and a rack holding region 3b for holding the racks 251 accommodating the test tubes 250 containing processed samples.

[Measuring Device 2 Structure]

The measuring device 2 is configured to be capable of obtaining optical information relating to a supplied sample by performing optical measurements of a sample supplied from the transporting device 3. In the present embodiment, optical measurements are performed on a sample dispensed into the cuvette 200 of the measuring device 2 from the test tube 250 held in the rack 251 of the transporting device 3.

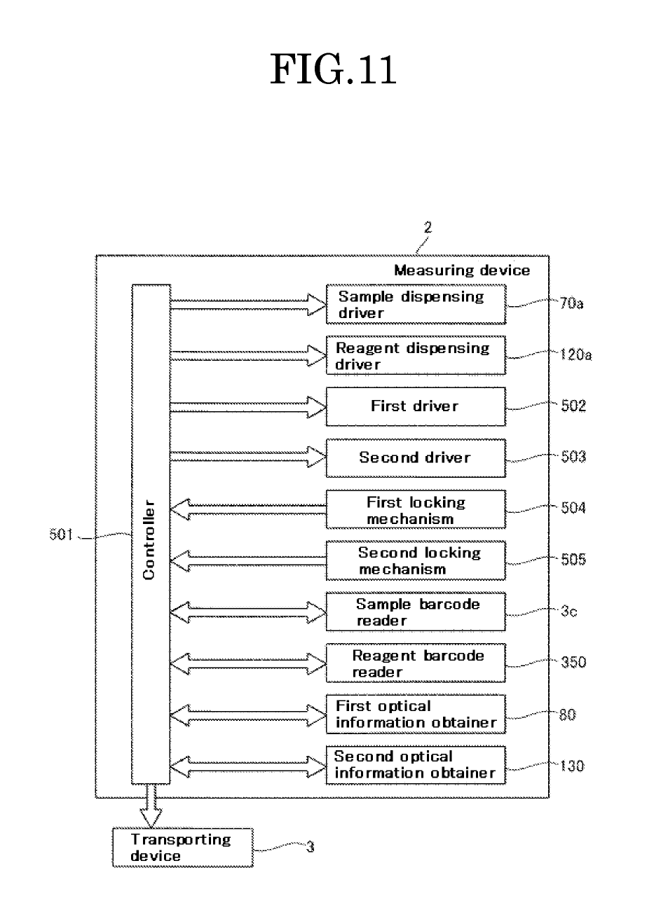

As shown in FIG. 11, the measuring device 2 has a sample dispensing driver 70a, reagent dispensing driver 120a, first driver 502, second driver 503, first lock detector 504, second lock detector 505, reagent barcode reader 350, sample barcode reader 3c, first optical information obtainer 80, second optical information obtainer 130, and controller 501 electrically connected to the transporting device 3.

The sample dispensing driver 70a is configured by a stepping motor with the function of vertically rotating a sample dispensing arm 70 (refer to FIGS. 3 and 5), a drive circuit for driving the stepping motor, and a pump or the like for aspirating and dispensing the sample (not shown in the drawings).

The reagent dispensing driver 120a is configured by a stepping motor with the function of vertically rotating a reagent dispensing arm 120 (refer to FIGS. 3 and 5), a drive circuit for driving the stepping motor, and a pump or the like for aspirating and dispensing the reagent (not shown in the drawings).

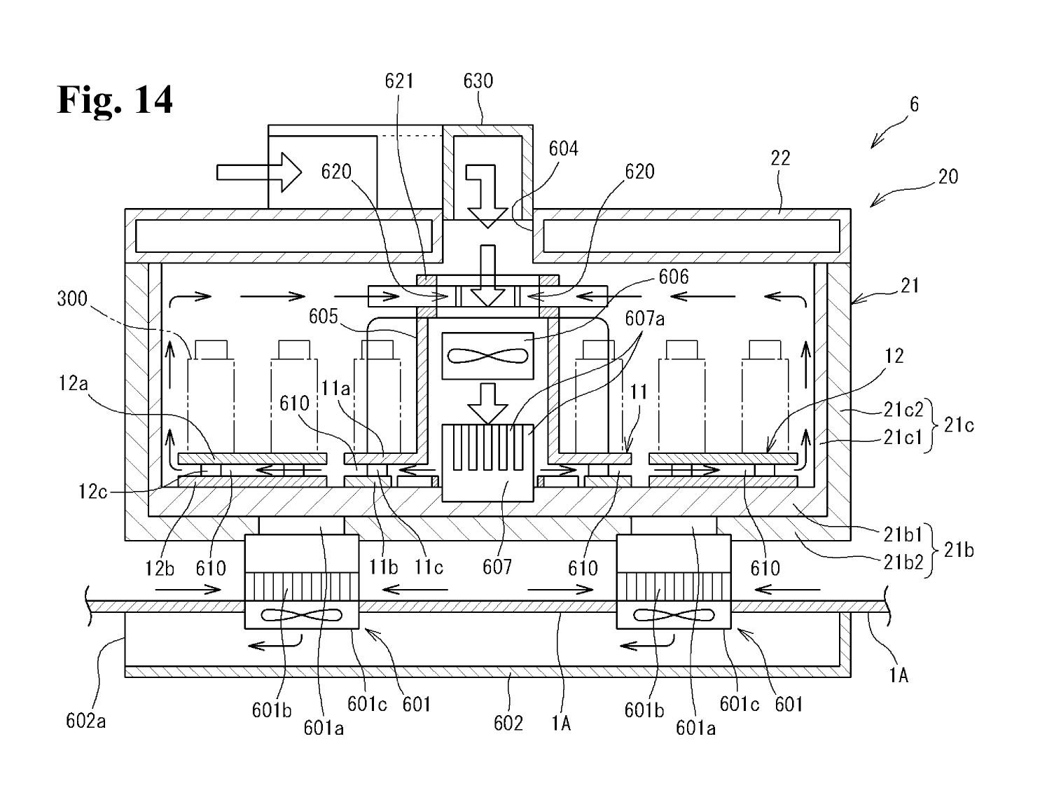

The first driver 502 is configured by a first stepping motor (not shown in the drawings) with the function of rotating a first reagent table 11 (to be described later; refer to FIGS. 5 and 14), and a drive circuit (not shown in the drawings) for driving the first stepping motor. The first reagent table 11 rotates and stops, the rotation being incremental in accordance with the number of pulses of a drive signal supplied from the controller 501 to the first driver 502.

Similarly, the second driver 503 is configured by a second stepping motor (not shown in the drawings) with the function of rotating a second reagent table 12 (to be described later; refer to FIGS. 5 and 14), and a drive circuit (not shown in the drawings) for driving the second stepping motor. The second reagent table 12 rotates and stops, the rotation being incremental in accordance with the number of pulses of a drive signal supplied from the controller 501 to the second driver 503.

Note that the controller 501 controls the rotational movement of the reagent tables 11 and 12 by determining the amount of movement of the reagent tables 11 and 12 from the origin position of the first reagent table 11 and the second reagent table 12 by counting the number of pulses of the supplied drive pulse signal.

The first lock detector 504 has the functions of detecting the lock state of a first cover 30 (to be described later; refer to FIG. 3), and transmitting a lock signal to the controller 501 when the first cover 30 is locked.

Similarly, the second lock detector 505 has the functions of detecting the lock state of a second cover 40 (to be described later; refer to FIG. 3), and transmitting a lock signal to the controller 501 when the second cover 40 is locked.

The reagent barcode reader 350 has the function of reading the barcodes of the first reagent table 11 and the second reagent table 12, and is disposed near the outer wall of the reagent reservoir 20 in the reagent storage section 6 to be described later, at a predetermined distance from the reagent reservoir 20 (refer to FIGS. 3 through 5). The reagent barcode reader 350 is capable of transmitting and receiving data to/from the controller 501, and has a drive circuit (not shown in the drawings) for controllably turning ON/OFF the reagent barcode reader 350. Note that the position of the reagent barcode reader 350 is normally fixed.

The sample barcode reader 3c has the function of reading the barcode adhered to the test tube 250 held in the rack 251 transported by the transporting device 3, and is disposed near the aspirating position 2a of the measuring device 2 and opposite the rack 251 transported by the transporting device 3 (refer to FIGS. 3 and 5). The sample barcode reader 3c is capable of transmitting and receiving data to/from the controller 501, and has a drive circuit (not shown in the drawings) for controllably turning ON/OFF the sample barcode reader 3c. Note that the position of the sample barcode reader 3c is normally fixed.

The first optical information obtainer 80 and the second optical information obtainer 130 (refer to FIGS. 3 and 5) have the function of obtaining optical information of the sample, and are configured to transmit and receive data to/from the controller 501.

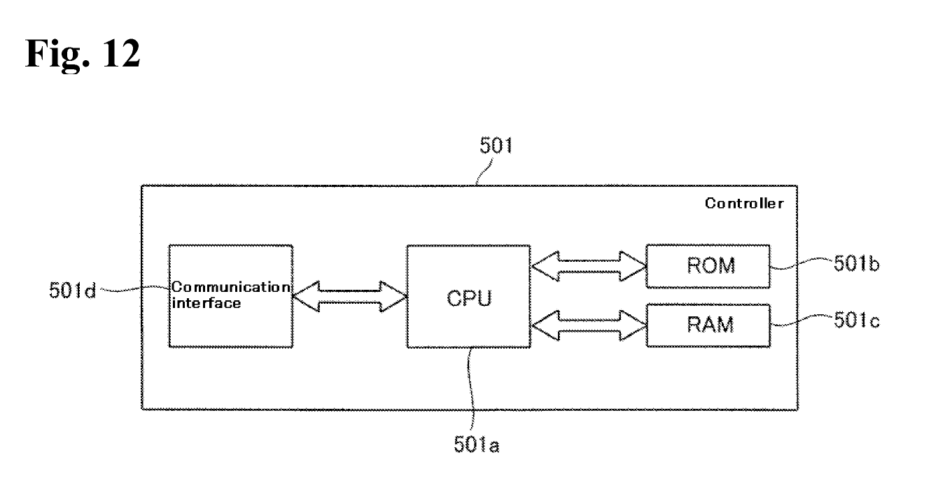

As shown in FIG. 12, the controller 501 is mainly configured by a CPU 501a, ROM 501b, RAM 501c, and communication interface 501d. The CPU 501a is capable of executing computer programs stored in the ROM 501b and computer programs read from the RAM 501c. The ROM 501b stores computer programs to be executed by the CPU 501a, and data and the like used in the execution of these computer programs. The RAM 501c is used when reading the computer programs stored in the ROM 501b. The RAM 501c is also used as the work area of the CPU 501a during the execution of the computer programs.

The communication interface 501d is connected to the control device 4, and has the functions of transmitting the optical information of a sample to the control device 4, and receiving signals from the controller 4a of the control device 4. The communication interface 501d also has the function of transmitting instructions from the CPU 501a for actuating each part of the transporting device 3 and measuring device 2.

As shown in FIG. 3, the measuring device 2 includes a reagent storage section 6 for storing reagent, and a reagent replacement section 7 for replacing or adding reagent. The reagent storage section 6 is provided to refrigerate at a low temperature (approximately 10° C.), and transport in a rotational direction, the reagent container 300 containing the reagent to be added to the sample within the cuvette 200. The reagent is prevented from degrading by storing the reagent at a low temperature. As shown in FIGS. 3 through 5, the reagent storage section 6 includes a reagent transporter 10 (refer to FIGS. 4 and 5) for holding and rotationally transporting the reagent, and a reagent reservoir 20 (refer to FIG. 3) disposed so as to cover the perimeter of the reagent transporter 10. The reagent transporter 10 holding the reagent is arranged in the refrigerated area formed within the reagent reservoir 20. Note that the specific structure and reagent cooling function of the reagent reservoir 20 in the reagent storage section 6 is described in detail later.

As shown in FIG. 5, the reagent transporter 10 includes a circular first reagent table 11, and an annular second reagent table 12 disposed concentrically with the first reagent table 11 on the outer side of the first reagent table 11. The first reagent table 11 and the second reagent table 12 are respectively configured so that the first reagent container rack 310 and the second reagent container rack 320 holding the reagent containers 300 are removable.

The first reagent table 11 and the second reagent table 12 are respectively rotatable in both clockwise and counterclockwise directions, and each table is rotatable so as to be mutually independent of the other. Thus, the first reagent container rack 310 and second reagent container rack 320 holding the reagent containers 300 containing the reagent are transported in a rotational direction by the respective first reagent table 11 and second reagent table 12. The reagent to be dispensed can be disposed near the reagent dispensing arm 120 by transporting the reagent container 300 in the rotational direction when the reagent dispensing arm 120 (described later) is to dispense the reagent.

As shown in FIG. 4, a shutter 21a which can open and close is provided on the side surface of the reagent reservoir 20 at a position facing the reagent barcode reader 350. The shutter 21a is configured to open only when the barcode reader 350 reads the barcodes of the reagent container 300, first reagent container rack 310, and second reagent container rack 320. Thus, the cold air within the reagent storage section 6 (refrigerated area) is prevented from escaping outside.

The reagent reservoir 20 is provided with a reagent reservoir body 21 (refer to FIGS. 4 and 5) formed as a cylinder with a bottom, and covers 22, 23, 30, and 40 (refer to FIG. 3) for covering the top opening of the reagent reservoir body 21; a space is formed within the reagent reservoir body 21 by the covers 22, 23, 30, and 40; and the reagent container 300 can be accommodated. The covers 22, 23, 30, and 40 are configured by a stationary cover 22 fixedly attached to the reagent reservoir body 21 so as to cover approximately the back half of the reagent reservoir body 21 and functions as a top wall of the reagent reservoir body 21, first and second covers 30 and 40 that are removable and cover approximately the right side front half of the reagent reservoir body 21, and a third cover 23 that is removable and covers approximately the left side front half of the reagent reservoir body 21. The stationary cover 22 is arranged within a housing 2A, and the third cover 23 is arranged within a cover body 2B disposed on the front side of the housing 2A. The first and second covers 30 and 40 are exposed on the right side of the cover body 2B, and configure the reagent replacement section 7 which is described later. The stationary cover 22, and first through third covers 30, 40, and 23 are divided front and back by the front wall 2A1 of the housing 2A.

As shown in FIG. 3, three holes 22a through 22c are formed in the stationary cover 22 of the reagent reservoir 20. Three holes 23a through 23c are also formed in the third cover 23 of the reagent reservoir 20. Aspiration of the reagent stored in the reagent storage section 6 is performed by the reagent dispensing arm 120 through the three holes 22a through 22c of the stationary cover 22. Aspiration of the reagent stored in the reagent storage section 6 is also performed by the sample dispensing arm 70 through the three holes 23a through 23c of the third cover 23. The sample dispensing arm 70 is configured to not only dispense the sample within the test tube 250 to the cuvette 200, but also to access the reagent reservoir 20 to dispense the reagent to the cuvette 200 and wash the pipette head.

Note that the holes 22a and 23a are positioned above the reagent container 300 held in the first reagent container rack 310. Reagent is aspirated from the reagent container 300 held in the first reagent container rack 310 through the holes 22a and 23a. The holes 22b and 22c, and holes 23b and 23c are respectively positioned above the reagent containers 300 held in the front row and back row of the second reagent container rack 320. Reagent is aspirated from the reagent containers 300 held in the front row and the back row of the second reagent container rack 320 through the holes 22b and 22c, and holes 23b and 23c.

The front side of the reagent reservoir 20 opens in an approximately semicircular mode by removing the third cover 23 together with the first cover 30 and second cover 40. The first reagent container rack 310 and second reagent container rack 320 are positioned within the reagent reservoir 20 through the opening when a measurement is started in the sample analyzer 1.

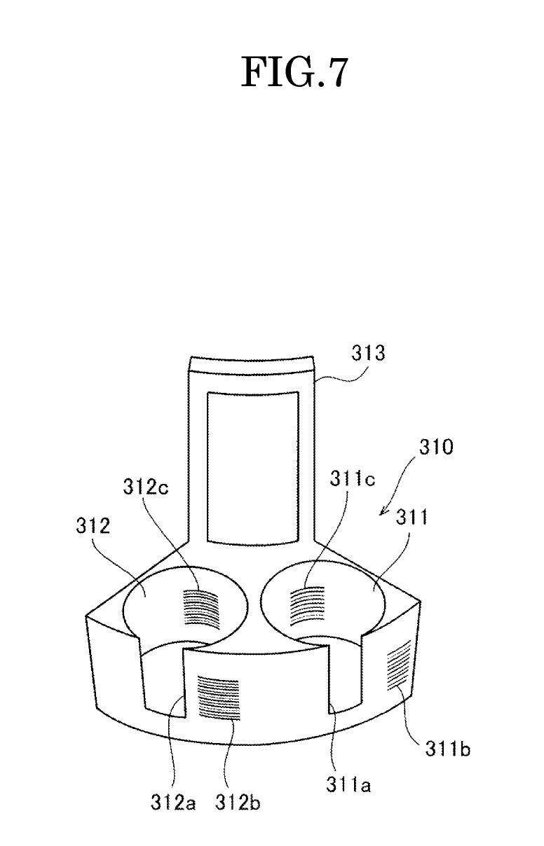

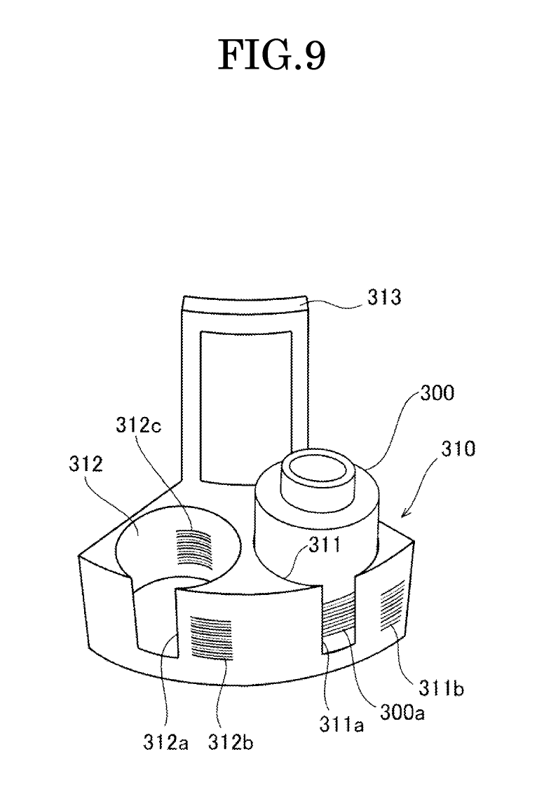

As shown in FIG. 5, five first reagent container racks 310 are deployable in the first reagent table 11. The reagent containers 300 are disposed in a ring in the five first reagent container racks 310. As shown in FIGS. 7 and 9, the first reagent container rack 310 includes two holders 311 and 312 for holding reagent containers 300, slots 311a and 312a respectively provided on the front side of the holders 311 and 312, and one handle 313 provided so as to project upward. As shown in FIG. 7, the holders 311 and 312 are circular in a planar view, can are capable of holding the reagent container 300 by inserting the cylindrical reagent container 300. The holders 311 and 312 can hold the reagent container 300, which has an external diameter that is smaller than the internal diameter of the holders 311 and 312, by attaching an adapter (not shown in the drawings) in the holders 311 and 312. The first reagent container rack 310 includes two types of racks so that the combinations of internal diameters of the holders 311 and 312 are different. A user can place reagent containers 300 of various sizes by changing the type of rack. Barcodes 311b and 312b are respectively provided on the front side of the holders 311 and 312, and barcodes 311c and 312c are respectively provided on the inside surface of the holders 311 and 312.

The two holder 311 and 312 can hold a plurality of individual reagent containers 300 that contain various reagents to be added when preparing a measurement sample from a specimen. That is, a maximum of ten (2×5=10) reagent containers 300 can be accommodated in the first reagent table 11. The slots 311a and 312a are provided to allow the reagent barcode reader 350 (refer to FIG. 5) to read the barcodes 311c and 312c. The holder 313 holds the first reagent container rack 310 when removing the rack from the reagent storage section 6.

The barcodes 311b and 312b include position information (holder number) for identifying the position of the holders 311 and 312. The barcodes 311c and 312c include information indicating the absence of a reagent container 300 in the holders 311 and 312 (reagent container absent information). The barcode 300a of the reagent container 300 includes information for specifying detailed information (reagent name, type of reagent container, lot number, reagent expiration date and the like) of the reagent contained in the reagent container 300.

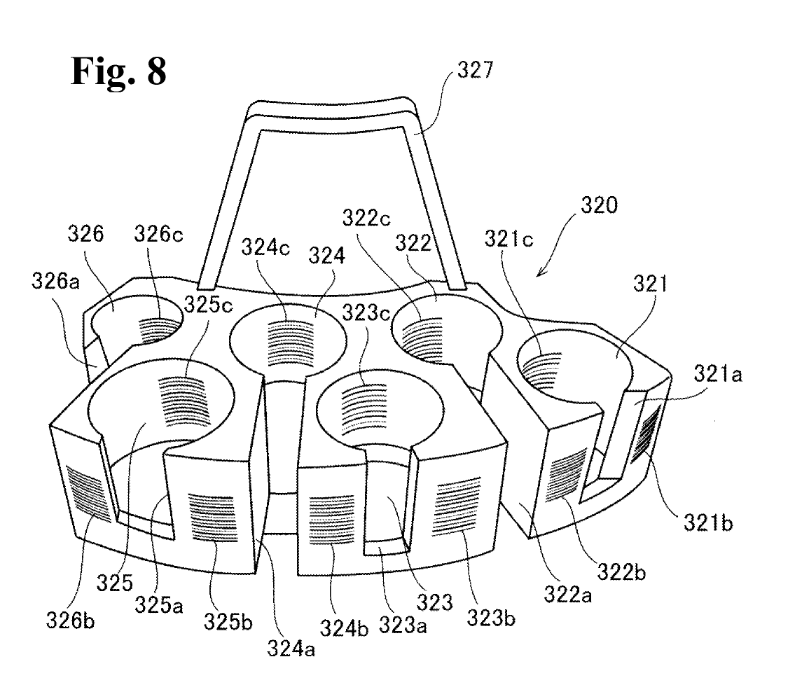

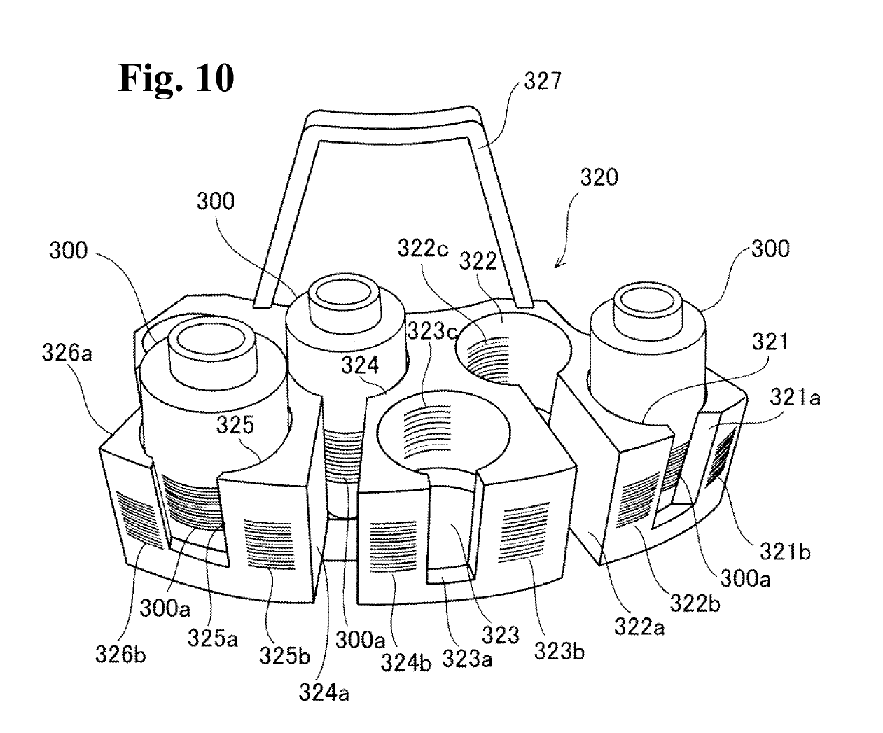

As shown in FIG. 5, five second reagent container racks 320 are deployable in the second reagent table 12. The reagent containers 300 are deployed in a ring shape in the five reagent container racks 320. One location among the gaps between the five places of the mutually adjacent second reagent container racks 320 has a space larger than the spaces of the other four locations. The barcodes 311b and 312b of the first reagent container racks 310 deployed in the first reagent container table 11 positioned on the inner side of the second reagent table 12, and the barcodes 300a of the reagent containers 300 held in the first reagent container racks 310 are read, through the largest gap 12a, by the barcode reader 350 positioned outside the reagent storage section 6. As shown in FIGS. 8 and 10, the second reagent container rack 320 includes six holders 321 through 326 for holding reagent containers 300, slots 321a through 326a respectively provided on the front side of the holders 321 through 326, and a handle 327 protruding from the top. Similar to the first reagent container rack 310, the holders 321 through 326 of the second reagent container rack 320 are circular in a planar view, and can hold the reagent container 300 when the cylindrical reagent container 300 is inserted. The second reagent container rack 320 includes three types of racks to provided different combinations of holders 321 through 326 with different internal diameters. The second reagent container rack 320 can accept deployment of the same reagents as the reagents deployed in the first reagent container rack 310.

Barcodes 321b and 322b are provided on bilateral sides of the slot 321a of the front row. Similarly, barcodes 323b and 324b, and barcodes 325b and 326b are respectively provided on bilateral sides of slots 323a and bilateral sides of slots 325a. Barcodes 321c through 326c are also provided on the inside surface of the holders 321 through 326.

The barcodes 321b through 326b respectively include position information (holder number) identifying the position of the holders 321 through 326. The barcodes 321c and 326c include information indicating the absence of a reagent container 300 in the holders 321 and 326 (reagent container absent information).

The controller 4a is configured to refer to the reagent master table, reagent lot master table, container master table and the like stored in the hard disk 401d based on the barcode information read by the reagent barcode reader 350, so as to obtain reagent identification information that includes the holder number, reagent name, lot number, reagent container type, reagent expiration date and the like. The obtained reagent identification information is then recorded in a reagent information database (not shown in the drawings) stored on the hard disk 401d. The information recorded in the reagent information database is reflected on the display 4b by the controller 4a of the control device 4.

As shown in FIGS. 1 and 2, the reagent replacement section 7 is provided near the center of the sample analyzer 1. In the present embodiment, the reagent replacement section 7 includes removable first cover 30 and second cover 40 respectively provided with the locking mechanisms 31 and 41, and a notifier 50 for notifying the user of the transport state of the first reagent table 11 and second reagent table 12, as shown in FIG. 3.

The first cover 30 is configured to be removable when replacing the reagent container 300 deployed in the first reagent table 11 (first reagent container rack 310). The locking mechanism 31 of the first cover 30 is provided to lock the first cover 30 during normal use or when the first cover 30 is mounted after reagent has been replaced or added, and to confirm to the controller 4a that the replacement or addition of reagent to the first reagent table 11 has been completed.

The second cover 40 is configured to be removable when replacing the reagent container 300 deployed in the second reagent table 12 (second reagent container rack 320). The locking mechanism 41 of the second cover 40 is provided to lock the second cover 40 during normal use or when the second cover 40 is mounted after reagent has been replaced, and to confirm to the controller 4a that the replacement or addition of reagent to the second reagent table 12 has been completed.

The notifier 50 includes two LED indicators 51 and 52. As shown in FIGS. 1 and 3, the two LED indicators 51 and 52 are positioned near the second cover 40 so as to be viewable by the user from outside the sample analyzer 1. The LED indicators 51 and 52 also can emit blue or red light.

The LED indicator 51 has the function of notifying the user that the first reagent container rack 310 corresponding to the user-specified reagent in the first reagent table 11 has been moved to the removal position (below the first cover 30) from which the reagent can be replaced. Specifically, the LED indicator 51 emits red light during the rotational movement of the first reagent table 11, and emits blue light when the first reagent container rack 310 corresponding to the user-specified reagent in the first reagent table 11 has been moved to the removal position and stopped. Thus, the notifier alerts the user to the timing for removing the first cover 30 to add or replace reagent.

The LED indicator 52 has the function of notifying the user that the second reagent container rack 320 corresponding to the user-specified reagent in the second reagent table 12 has been moved to the removal position (below the second cover 40) from which the reagent can be replaced. Similar to the LED indicator 51, the LED indicator 52 emits red light during the rotational movement of the second reagent table 12, and emits blue light when the second reagent container rack 320 corresponding to the user-specified reagent in the second reagent table 12 has been moved to the removal position and stopped.

After the reagent has been added or replaced and the user has locked the first cover 30 or the second cover 40, the sample analyzer 1 automatically reads the barcodes 300a of all reagent containers 300 held in the first reagent container rack 310 or second reagent container rack 320 in which the reagent was replaced. Thus, when, the reagent deployment is accurately managed after replacement even when, for example, a single reagent has been specified and reagent replacement has been instructed, but reagents other than the specified reagent also have been replaced in the same first reagent container rack 310 or second reagent container rack 320 in addition to the specified reagent.

As shown in FIGS. 3 through 5, the measuring device 2 is provided with a cuvette transporter 60, sample dispensing arm 70, first optical information obtainer 80, lamp unit 90, heater 100, cuvette mover 110, reagent dispensing arm 120, second optical information obtainer 130, urgent sample placer 140, fluid unit 150, and cuvette supplier 160.

[Reagent Reservoir 20 Structure and Reagent Cooling Function]

The specific structure and reagent cooling function of the reagent reservoir 20 of the reagent storage section 6 is described in detail below. FIG. 14 is a cross sectional view schematically showing the reagent reservoir 20. The reagent reservoir 20 is provided with a reagent reservoir body 21 formed as a cylinder with a bottom, and covers (stationary cover 22, first through third covers 30, 40, and 23) for closing the top openings of the reagent reservoir body 21; and has a space formed within the reagent reservoir body 21 by the covers 22, 23, 30, and 40 so as to accommodate the reagent container 300.

The bottom wall 21b and perimeter wall 21c of the reagent reservoir body 21 are respectively configured as internal-external two-layer structures, wherein the internal layers 21b1 , 21c1 are thermal transfer layers formed of material that has a thermal conductivity such as aluminum and the like. The outer layers 21b2 and 21c2, on the other hand, are heat insulating layers formed of material, such as synthetic resin or the like, that has lower thermal conductivity than the internal layers 21b1 and 21c1. The covers 22, 30, 40, and 23 are also heat insulating layers formed of material, such as synthetic resin or the like, that has lower thermal conductivity than the internal layers 21b1 and 21c1.

The inner layer 21b1 of the bottom wall 21b of the reagent reservoir body 21 is partially exposed on the bottom side, and the exposed surface is provided with one or more (two in the example of the drawing) of coolers 601. The cooler 601 of the present embodiment uses a Peltier element 601a, a heat sink 601b is provided on the bottom surface (heat emitting side) of the Peltier element 601a, and a heat radiating fan 601c is also provided on the bottom surface of the heat sink 601b. The cooler 601 is configured to cool the air within the reagent reservoir 20 using the body of the inner layer 21b1 itself as a cooling medium by directly cooling the inner layer 21b1 of the reagent reservoir body 21 with high thermal conductivity. Note that the cooler 601 is not limited to using a Peltier element 601a, inasmuch as the inner layers 21b1 and 21c1 may also be cooled, for example, by cold air or cold water.

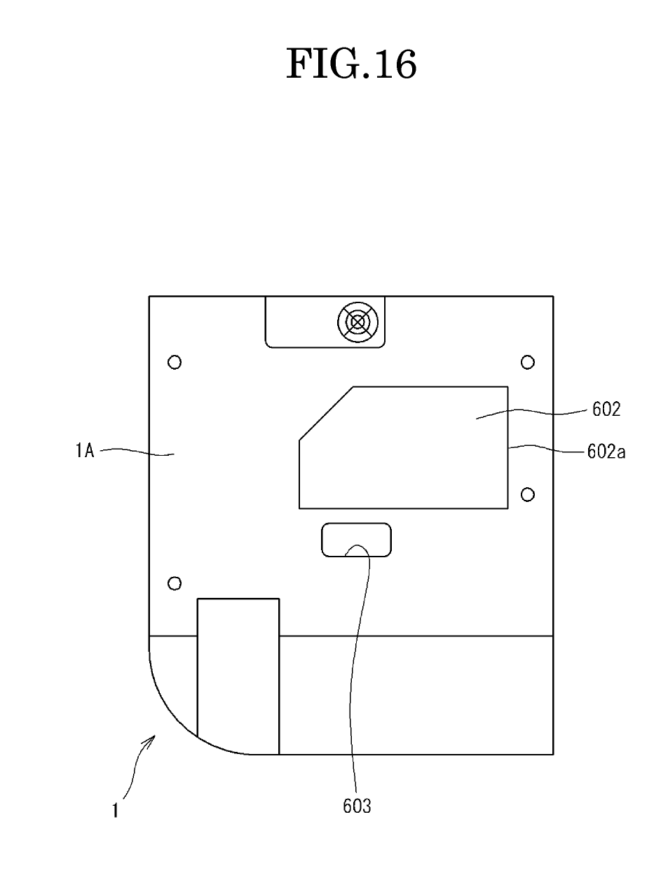

The heat radiating fan 601c is configured to expel hot air from the exhaust outlet formed in the bottom surface 1A of the sample analyzer 1 after the air within the housing 2A of the sample analyzer 1 has been aspirated to the heat sink 601b and heat exchange has occurred by the heat sink 601b. An exhaust duct 602 for expelling the hot air is also provided on the bottom surface 1A of the sample analyzer 1. FIG. 16 shows the bottom surface of the sample analyzer 1; the exhaust duct 602 provided in the bottom surface 1A of the sample analyzer 1 faces laterally on the sample analyzer 1. An aspiration hole 603 for aspirating the radiant heat air is formed on the front side of the exhaust duct 602. Directly aspirating exhaust air to the aspirating hole 603 can be prevented and having hot air expelled toward the user operating the front of the sample analyzer 1 can be avoided by having the exhaust outlet 602a of the exhaust duct 602 face laterally.

As shown in FIGS. 3 and 14, an air induction port 604 is provided in the center of the top surface of the reagent reservoir 20 to take in the air within the housing 2A into the reagent reservoir 20. Specifically, the air induction port 604 passes vertically through the stationary cover 22. According to this configuration, the air within the housing 2A can be taken into the reagent reservoir 20 through the air induction port 604. A cylindrical flow tube 605, which forms a flow path for the air inducted from the air induction port 604, is provided directly below the air induction port 604; the flow tube 605 is provided with a fan 606 for blowing the air inducted from the air induction port 604 downward into the flow tube 605, and promoting the induction of air through the air induction port 604. The operation of the fan 606 actively inducts the air within the housing 2A through the air induction port 604 and into the reagent reservoir 20, and blows the air within the flow tube 605 downward and subsequently expels the air from the bottom end of the flow tube 605 and throughout the entirety of the reagent reservoir 20.

The bottom end of the flow tube 605 is integratedly formed with the first reagent table 11, so as to rotate around with the first reagent table 11. The top surfaces (reagent mount) 11a and 12a of the first reagent table 11 and second reagent table 12 are formed by material of low thermal conductivity such as synthetic resin or the like, and the bottom surfaces 11b and 12b of the reagent tables 11 and 12 are formed of material that has a higher thermal conductivity than the top surfaces 11a and 12a, such as aluminum or the like. An air flow gap 610 is formed via a spacer 11c between the top surface 11a and bottom surface 11b, and a spacer 12c between the top surface 12a and bottom surface 12b. The air flow gap 610 communicates with the interior of the flow tube 605, so that air inducted from the air induction port 604 flows through the flow tube 605 and to the gap 610. Since the top surfaces 11a and 12a of the first and second reagent tables 11 and 12 are formed of material of low thermal conductivity, the reagent containers 300 on the first and second reagent tables 11 and 12 are slightly cooled directly by the cold air flowing through the gap 610 and the entirety of the reagent reservoir 20 is cooled by the flowing cold air.

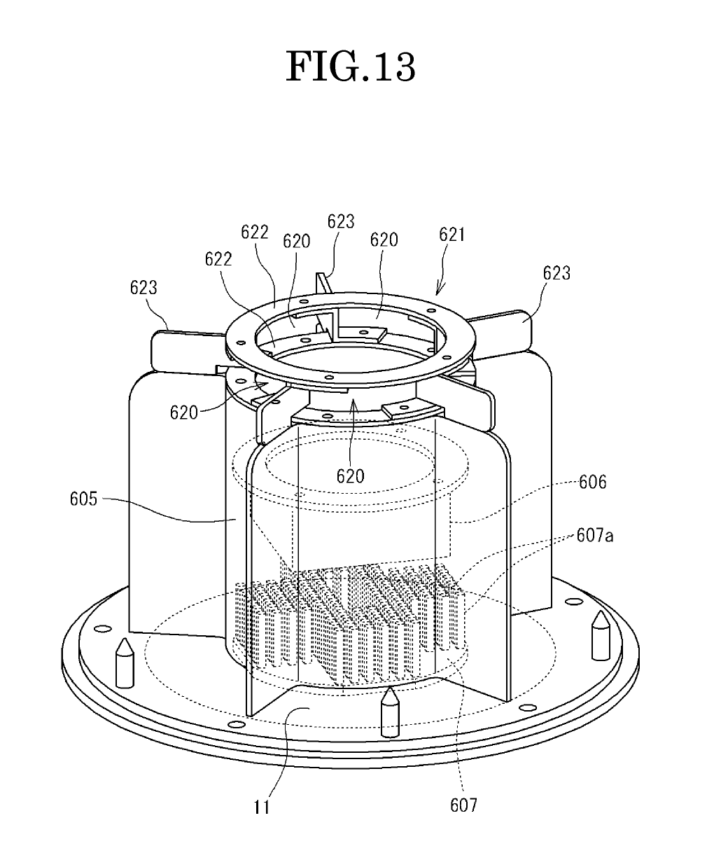

In the flow tube 605, a dew condensation promoting block (a dew condensation promoter material) 607, which is formed of material of high thermal conductivity such as aluminum or the like, is provided below the fan 606. As shown in FIG. 13, the dew condensation promoting block 607 is provided with a plurality of rows of many upward facing rod-like projections 607a. The dew condensation promoting block 607 is provided in contact with the inside layer 21b1 of the bottom wall 21 of the reagent reservoir 20. Therefore, the dew condensation promoting block 607 is also cooled by the cooler 601, and the air within the reagent reservoir 20 is cooled as a cooling medium.

The air inducted through the air induction port 604 by the fan 606 is blown directly to the dew condensation promoting block 607, and the excess moisture is eliminated when the water vapor contained in the air condenses on the dew condensation promoting block. The dew condensation promoting block 607 in particular further promotes dew condensation by increasing the surface area in contact with the air through the plurality of rod-like projections 607a.

The air blown on the dew condensation promoting block 607 flows through the gap 610 in the diameter direction to the outside of the reagent reservoir 20, then flows upward along the inner wall 21c1 of the perimeter wall 21c. This flow further cools the air within the reagent reservoir 20 via the inside wall 21c1, and the cold state is maintained. The air that reaches the top of the perimeter wall 21c then flows in the diameter direction toward the inner side along the bottom surface of the cover 22. The entirety of the interior of the reagent reservoir 20 is thus cooled by the air flow. The air within the reagent reservoir 20 again reaches the top of the flow tube 605 and circulates from a circulation port 620 formed on the top of the flow tube 605 into the flow tube 605.

Specifically, a circulation member 621 provided with the circulation port 620 is formed in the top of the flow tube 605. As shown in FIG. 13, the circulation member 621 is configured by a pair of top and bottom ring bodies 622 with a central opening, and guide fins 623 deployed radially between the pair of top and bottom ring bodies 622, and the circulation port 620 is provided medially to the pair of top and bottom ring bodies 622, and between the guide fins 623. The air that has been inducted through the air induction port 604 and flowed within the reagent reservoir 20, then flows into the circulation port 620 and is blown, together with the fresh air inducted through the air induction port 604 onto the dew condensation promoting block 607. The temperature within the reagent reservoir 20 is rapidly equalized and the cooling efficiency is improved by circulating within the flow tube 605 the low temperature air which has been cooled by the flowing within the reagent reservoir 20.

Note that part of the air flowing in the reagent reservoir 20 is expelled from the holes 22a through 22c and holes 23a through 23c formed in the covers 22 and 23 of the reagent reservoir 20, thereby balancing the air pressure within the reagent reservoir 20, as shown in FIG. 3.

As shown in FIG. 3, the air induction port 604 is disposed further to the front side of the housing 2A than the front wall 2A1, and is connected to the intake duct (flow path member) 630 to induct the air within the housing 2A (further to the back side than the front wall 2A1). The intake duct 630 extends backward from the air induction port 604 and passes through the front wall 2A1 on the top surface of the cover 22 of the reagent reservoir 20. The intake duct 630 is L-shaped to bend to the opposite side (left side) of the reagent aspirating holes 22a through 22c formed in the stationary cover 22. The intake duct 630 is disposed between the air induction port 604 and the holes 22a through 22c formed in the stationary cover 22, and functions as a flow blocker to prevent the flow of the air within the reagent reservoir 20 from flowing directly to the air induction port 604 immediately after being discharged from the holes 22a through 22c.

The intake duct 630 is provided for the following reasons. When the intake duct 630 is not provided on the air induction port 604, the air expelled from the holes 22a through 22c is actively aspirated by the nearby air induction port 604, creating a narrow range of air circulation inside and outside the reagent reservoir 20 between the air induction port 604 and the holes 22a through 22c. When such circulation is created, it becomes difficult to expel air from the other holes 23a through 23c, and the flow of air within the reagent reservoir 20 becomes unbalanced and causes uneven temperatures within the reagent reservoir. Therefore, providing the intake duct 630 produces a balanced air discharge from the holes 22a through 22c and holes 23a through 23c so as to create a uniform temperature within the reagent reservoir 20.

The intake duct 630 also has the function of preventing light from outside the analyzer 1 from entering from the openings of the reagent reservoir 20 into the housing 2A through the air induction port 604 and reaching the optical information obtainer 130. That is, the intake duct 630 functions as a light shield for blocking the light between the air induction port 604 and the optical information obtainer 130.

In the present embodiment, there is no need for a large temperature differential between the set reagent temperature (target temperature) and the temperature of the inside layers 21b1 and 21c1 of the reagent reservoir 20 due to the cooling of the interior (inside layers 21b1, 21c1) of the reagent reservoir 20 by the cooler 601, and the uniform low temperature condition inside the reagent reservoir 20 created by the flow (circulation) of air within the reagent reservoir 20. Specifically, the inside layers 21b1 and 21c1 of the reagent reservoir 20 may be cooled by the cooler 601 to a low temperature that is 2 to 3° C. lower than the reagent set temperature (target temperature). Therefore, the air within the reagent reservoir 20 is not overly cooled, and a suitable temperature can be maintained within the reagent reservoir 20 thus preventing the reagent from drying out.

That is, the temperature of the cold air must be reduced below the target temperature to achieve the target temperature of the reagent when cold air from a place other than the reagent reservoir is introduced into the reagent reservoir and circulated to cool the reagent. Although the humidity within the reagent reservoir is thus reduced and drying of the reagent is promoted with the possibility of adversely affecting the reagent components, these problems do not occur in the present embodiment.

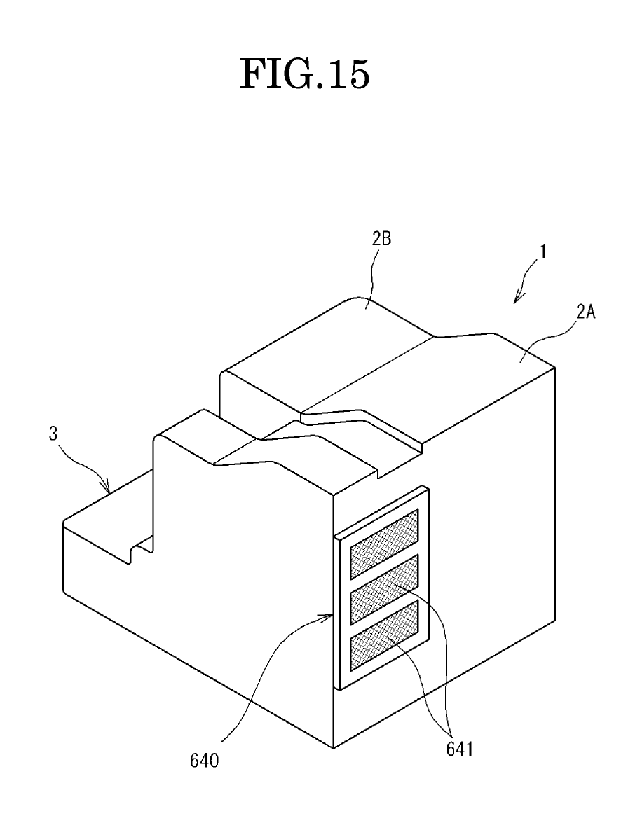

FIG. 15 is a perspective view of the back side of the sample analyzer 1. An outside air intake port 640 is provided on the back side of the analyzer 1, and a filter 641 is installed in the outside air intake port 640. Therefore, clean air from which dirt has been removed by the filter 641 flows into the housing 2A of the sample analyzer 1, and the clean air is inducted into the reagent reservoir 20 from the air induction port 604. Note that the filter 641 may also be provided on the air induction port 604 and intake duct 630, but providing the filter 641 on the housing 2A is desirable due to the complexity cleaning and replacing the filter 641 when installed in the housing 2A.

The sample analyzer of the present embodiment described above is configured to cool the interior of the sample analyzer 1 through the air induction port 604, and more specifically air is introduced into the housing 2A and the introduced air is cooled by the inside layer 21b1 of the reagent reservoir 20 cooled by the Peltier element 601a. Thus, excess air is prevented from entering the reagent reservoir 20 and dew condensation is prevented because the adverse effects of outside airflow from the laboratory is not incurred. Note that the air within the sample analyzer 1 is normally relatively warm compared to outside air due to the influence of the devices operating within the analyzer, so that dew condensation readily occurs if the air is cooled. The inventors of the present invention discovered that dew condensation can be prevented if the analyzer is configured so that the air within the analyzer is introduced into the reagent reservoir regardless of the situation mentioned above.

[Reagent Replacement and Addition Operation]

The operation of adding and replacing the reagent container 300 in the reagent reservoir 20 of the reagent storage section 6 is accomplished by opening the first cover 30 and second cover 40 which configure the reagent replacement section 7; in this case the cooler 601 that cools the inside layer of the reagent reservoir 20 and the fan 606 disposed within the reagent reservoir 20 are turned OFF. Specifically, when the controller 501 confirms that the locking mechanisms 31 and 41 are unlocked to open first and second covers 30 and 40, the controller 501 stops the operation of the fan 606 and cooler 601. Thus, excess air flow is prevented within the reagent reservoir 20, and dew condensation of water vapor in outside air flowing into the reagent reservoir 20 is prevented by opening the first and second covers 30 AND 40.

The present invention is not limited to the embodiment described above, and may be variously modified insofar as such modification is within the scope of the claims. For example, although air is circulated within the reagent reservoir 20 by a fan 606 in the sample analyzer 1 of the above embodiment, the present invention is not limited to this arrangement inasmuch as the fan 606 may be omitted and cold air may descent within the reagent reservoir to circulate the air if the Peltier element 601a is provided on the top surface (for example, cover 22) of the reagent reservoir 20. Although the air induction port 604 is provided on the stationary cover 22 of the reagent reservoir 20 in the above embodiment, the air induction port 604 may also be formed on a removable cover (first through third covers 30, 40, 23).

The present invention is not limited to the reagent reservoir used in coagulation analyzers such as that of the above embodiment, and may also be applied to reagent reservoirs holding reagent containers used in biological analyzers such as immunoanalyzers and the like.

What is claimed is:

1. An analyzer comprising:
a housing comprising a first space therein;
a reagent accommodation section, disposed in the housing, comprising a second space therein for accommodating a reagent container containing a reagent, and an air induction port for inducting air from the first space to the second space; and
a cooler for cooling the air which has been inducted from the first space through the air induction port into the second space.

2. The analyzer of claim 1 further comprising
a flow unit for flowing the air which has been inducted into the second space through the air induction port.

3. The analyzer of claim 2, wherein
the flow unit promotes the air within the first space to enter the second space through the air induction port.

4. The analyzer of claim 1, wherein
an inside surface of the reagent accommodation section is formed of a thermally conductive material; and
the cooler cools the inside surface of the reagent accommodation section.

5. The analyzer of claim 4, wherein
an outside surface of the reagent accommodation section is formed of a material with a thermal conductivity lower than that of the material that forms the inside surface of the reagent accommodation section.

6. The analyzer of claim 4, wherein
a temperature differential between a target temperature of the reagent and a temperature of the inside surface of the reagent accommodation section cooled by the cooler is set at 3° C. or less.

7. The analyzer of claim 1, wherein
the reagent accommodation section comprises:
a main body comprising an opening at a top end thereof and accommodating the reagent container received through the opening; and
a cover removably covering the opening at the top end of the main body and comprising the air induction port.

8. The analyzer of claim 7, wherein
an inside surface of the reagent accommodation section is formed of a thermally conductive material; and
the cover is formed of a material with a thermal conductivity lower than that of the material that forms the inside surface of the reagent accommodation section.

9. The analyzer of claim 7 further comprising:
a flow unit for flowing the air which has been inducted into the second space through the air induction port; and
a controller for stopping an operation of the flow unit when the cover is removed.

10. The analyzer of claim 1, wherein
a reagent holder for holding the reagent container is disposed inside the reagent accommodation section.

11. The analyzer of claim 10, wherein
the reagent holder comprises a first member for holding the reagent container, a second member contacting an inside surface of the reagent accommodation section, and a third member for forming a space between the first member and the second member; and
the first member is formed of a thermally conductive material, and the second member is formed of a material with a thermal conductivity lower than that of the material forming the first member.

12. The analyzer of claim 2 further comprising
a dew condensation promoter for promoting dew condensation of the air flowed by the flow unit.

13. The analyzer of claim 12, wherein:
the flow unit blows downward the air within the second space; and
the dew condensation promoter is disposed in a blowing direction of the air by the flow unit within the second space.

14. The analyzer of claim 12, wherein
the dew condensation promoter comprises a plurality of rod-like members extending in a blowing direction of the air by the flow unit.

15. The analyzer of claim 1, wherein
the housing comprises an outside air intake comprising an air intake opening and a filter covering the air intake opening.

16. The analyzer of claim 1, wherein
the reagent accommodation section comprises a reagent aspiration port for aspirating the reagent within the reagent container; and
the analyzer further comprises a flow blocking member for preventing air from flowing between the air induction port and the reagent aspirating port.

17. The analyzer of claim 1 further comprising
a flow tube for forming a flow path of the air which has been inducted into the second space through the air induction port.

18. The analyzer of claim 17 further comprising
a reagent holder for holding the reagent container, which is formed so as to circumscribe an outer surface of the flow tube.

19. The analyzer of claim 1, wherein
the reagent is a coagulation reagent used for measuring an item related to a coagulation function of a blood specimen.
